(19) United States
(12) Patent Application Publication
(10) Pub. No.: US 2021/0323699 A1
(43) Pub. Date: Oct. 21, 2021

(54) AIRCRAFT DISINFECTING SYSTEM

(71) Applicant: Charles Thomas Myers, Myrtle Beach, SC (US)

(72) Inventor: Charles Thomas Myers, Myrtle Beach, SC (US)

(21) Appl. No.: 17/230,285

(22) Filed: Apr. 14, 2021

Related U.S. Application Data
(60) Provisional application No. 63/010,107, filed on Apr. 15, 2020.

(51) Int. Cl. B64F 5/30; A61L 2/22

(52) U.S. Cl. CPC B64F 5/30; A61L 2/22; B64D 13/00; A61L 2202/25; A61L 2202/15

(57) ABSTRACT

An aircraft disinfecting system for use in an aircraft fuselage having a frame, an inner shell, and an outer shell the includes a source of disinfecting solution; plumbing inside of the aircraft fuselage connected to the disinfecting solution source for transferring the disinfecting solution; disinfecting solution dispersers connected to the plumbing located inside of the aircraft for applying disinfectant to surfaces to be disinfected; and a controller for regulating the disinfecting system for applying the disinfectant throughout the aircraft fuselage.

### CROSS-REFERENCE TO RELATED APPLICATIONS

The contents of Provisional Application U.S. Ser. No. 63/010,107 filed Apr. 15, 2020, on which the present application is based, and benefit claimed under 35 U.S.C. § 119(e), is herein incorporated by reference.

### BACKGROUND OF THE INVENTION

### 1. Field of the Invention

The present invention relates to disinfecting the interior of an aircraft fuselage. More particularly, the present invention relates to embodiments of a disinfecting system that is built into an aircraft fuselage between the outer and inner shells and which system is easily adapted to a variety of aircraft.

### 2. Description of the Prior Art

The fuselage of conventional aircraft accommodates passengers, baggage, and, depending upon the size, cargo. The fuselage has a frame with an outer shell on the outside and an inner shell on the inside of the frame. The design and size of the fuselage determines the number of passengers that are accommodated but in any event the passengers are seated rather closely together. Under such circumstances the passengers are susceptible to a passenger that make have an unhealthy condition and when deplaning germs and whatnot from the unhealthy passenger are left behind on seats and the like for the onboarding passengers to make contact and thereby become infected.

It is important for aircraft to be disinfected to prevent the spread of viruses. Following each flight it is best practices to ensure the aircraft is fully disinfected. The airlines have established procedures for cleaning and disinfecting aircraft. These procedures follow guidelines from the Center for Disease Control and Prevention and are necessary for ensuring that aircraft are free from viruses and the like. Cleaning crews presently use routine cleaning and disinfecting the interior of the fuselage with detergent and/or disinfecting solution for cleaning the aircraft. These procedures, currently in use, include spraying and wiping down seats and the like, which is obviously labor intensive and time consuming.

### SUMMARY OF THE INVENTION

There is provided several embodiments of a disinfecting system for use by an aircraft cleaning crews to disinfect the interior of the fuselage of the aircraft more easily and effectively than the presently used procedures. The disinfecting systems of the present invention include a source of disinfecting solution supplied to an aircraft. Plumbing is located between the outer and interior shell of the aircraft fuselage passing through the frame and connecting to the disinfecting solution source for transferring the disinfecting solution. Dispersant nozzles are connected to the other end of the plumbing for applying disinfecting solution to surfaces to be disinfected. There is also provided a controller for operating the disinfecting system to regulate the supply of disinfectant applied throughout the aircraft fuselage. In a first embodiment an inlet port on the external side of the aircraft fuselage allows the cleaning crew to connect to an external disinfectant source. This embodiment of the disinfecting system includes outlet nozzle(s) installed in different locations inside of the aircraft through which the disinfectant is applied. In other embodiments, the dispersant outlet nozzles are connected through the aircraft ventilation system. In yet another embodiment of the dispersing system of the present invention an internal holding tank that contains disinfectant is installed within the aircraft and connects through the plumbing. The disinfectant is applied through outlet nozzles throughout the plane or through the ventilation system. The disinfectant system of the present invention is easily adaptable to a variety of aircraft fuselages.

It is the general object of this invention to provide a disinfecting system which automates some of the presently established disinfecting procedures.

Another object of the present invention is to provide a disinfecting system having several embodiments that make it adaptable to many fuselage designs and sizes.

Other objects, features and advantages of the present invention will become apparent to those skilled in the art from the following detailed description of the invention taken in conjunction with the accompanying drawings.

### BRIEF DESCRIPTION OF THE DRAWING(S)

Having described the invention in general terms, reference will be made to the accompanying drawing(s), which are not necessarily drawn to scale.

FIG. 1 is a cross-sectional view of an aircraft fuselage illustrating a first embodiment of the disinfectant system of the present invention showing an inlet port on the outside of the aircraft fuselage for connecting to a source of disinfecting solution to the aircraft and applying the disinfectant through the aircraft ventilation system;

FIG. 2 is a cross-sectional view of an aircraft fuselage illustrating a second embodiment of the disinfectant system of the present invention showing a holding tank for holding disinfectant located on the inside of an aircraft fuselage and connected through plumbing and applying the disinfectant through the aircraft ventilation system;

FIG. 3 is a cross-sectional view of an aircraft fuselage illustrating yet another embodiment of the disinfectant system of the present invention showing an inlet port located on the side of an aircraft fuselage for connecting to a source of disinfecting solution located outside of the aircraft to the plumbing/controls, and the plumbing connected to dispersant nozzles; and

FIG. 4 is a cross-sectional view of an aircraft fuselage illustrating another embodiment of the disinfectant system of the present invention showing a holding tank for holding disinfecting solution located on the inside of an aircraft fuselage for holding disinfecting solution and connected through plumbing to dispersant nozzles.

### DESCRIPTION OF THE PREFERRED EMBODIMENTS

The present invention now will be described more fully hereinafter with reference to the accompanying drawings, in which preferred embodiments of the invention are shown. This invention may, however, be embodied in several different forms and should not be construed as limited to the embodiments set forth herein; rather these embodiments are provided so that this disclosure will be through and complete and will fully convey the scope of the invention to those skilled in the art.

The present invention is adaptable to large or small aircraft of differing designs. The fuselages of the aircraft for which the present invention is useful are typically built around a frame and have an outer shell and an inner shell and house the passengers. The size and style of the aircraft will in part determine which of the embodiments will be used on a particular aircraft.

In general, this invention provides several embodiments of a system to simplify disinfecting the interior of an aircraft fuselage. A source of disinfecting solution is supplied to the aircraft; plumbing inside of the aircraft fuselage between said inner shell and said outer shell is connected to the disinfecting solution source. Disinfecting solution dispensers are connected to the other end of the plumbing and are located inside of the aircraft for applying disinfecting solution to surfaces to be disinfected. A controller is connected to the disinfecting system for controlling the amount of disinfectant applied throughout the aircraft fuselage.

In a first embodiment there is provided an inlet port 110 on the outside of the outer shell of the aircraft fuselage 100 that connects to an external disinfecting solution supply, such as a supply truck containing the disinfecting solution. The system includes disinfecting dispenser, outlet dispensing nozzles 120 installed in different locations inside of the aircraft fuselage ventilating system 118 to apply the disinfecting solution. In FIG. 1, there is shown a cross-sectional view of an aircraft fuselage 100 showing the fuselage outer shell 102 and the inner shell 104. In this embodiment there is an inlet port 110 located on the outside of the aircraft fuselage for making a connection to a source of disinfecting solution. Disinfecting solution dispersant nozzles 120 are located inside the existing aircraft ventilation system 118. The inlet port 110 and the dispersing nozzles 120 are connected to each other through the appropriate plumbing 130 that extends between the inner shell 102 and outer shell 104. The plumbing is made of flexible piping and has typical connectors connecting the inlet port 110 and the dispersing nozzles 120 to the plumbing. The disinfecting system is controlled through a controller 106 that may be located at various positions along the route of the plumbing, depending upon the aircraft. Appropriate controls, such as on/off switches, pumps and the like, known to those skilled in the art, are provided in controller 106 to transport the disinfecting solution through the system control the various nozzles and valves associated therewith. When the system is operating the dispersant nozzles 120 apply a mist of disinfecting solution throughout the aircraft fuselage. By way of example, the fuselage 100 shown in FIG. 1 is that of a Fokker having a configuration of four seats across the fuselage interior.

This embodiment allows the disinfecting solution to be applied throughout the interior of the aircraft without the cleaning crew having to carry hoses and the like throughout the aircraft to dispense the disinfecting solution. The disinfecting solution may be left to air dry. Optionally, as part of the disinfecting system the cleaning crew may use an electrostatic sprayer as an adjunct of the system. The electrostatic sprayer delivers an electric charge to the disinfectant to help the droplets adhere to surfaces.

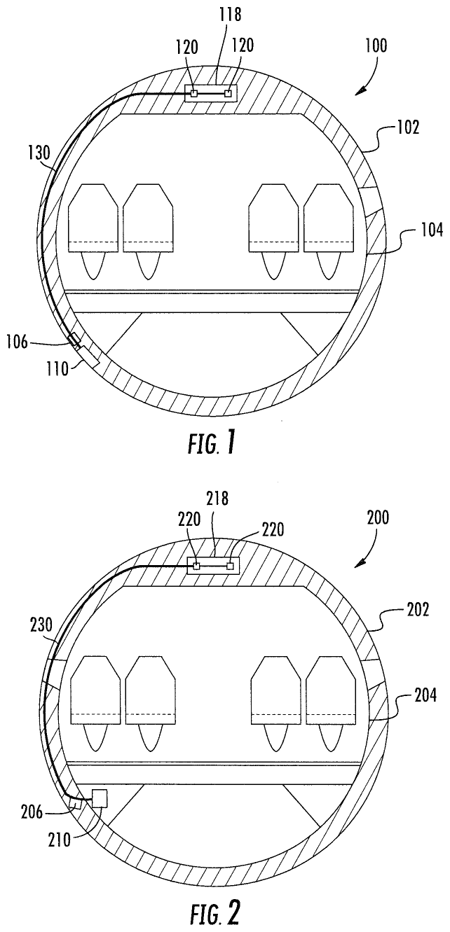

There are situations airports may not have available an external source of disinfecting solution. In such situations the aircraft may utilize a built-in holding tank for the disinfecting solution, such as the embodiment shown in FIG. 2. In FIG. 2 there is a shown a cross-sectional view of an aircraft fuselage 200 having an outer shell 202 and an inner shell 204 and illustrating the disinfecting system located within the aircraft fuselage. Holding tank 210 is located within the fuselage 200 and is connected through appropriate plumbing 230 to dispersing nozzles 220 located throughout the inside of the aircraft ventilation system 218. Controls, such as on/off switches, pumps and the like, known to those skilled in the art, are provided in controller 206 to transport the disinfectant and control the various nozzles and valves associated with the system. The embodiment, like the others allows the dispersant mist to be applied throughout the interior of the aircraft without the cleaning crew having to carry hoses and the like throughout the aircraft in order to dispense the disinfectant.

The disinfecting system of the embodiment shown in FIG. 3 varies somewhat from the embodiments of FIG. 1 and FIG. 2 in that rather than using the existing aircraft ventilation system for supplying disinfecting solution separate nozzles are used. Thus, there is shown in FIG. 3 a cross-sectional view of an aircraft fuselage 300 having an outer shell 302 and an inner shell 304 and illustrating another embodiment of the disinfecting system of the present invention. There is an inlet port 310 located on the outside of aircraft fuselage 300 capable of making a connection to disinfecting solution sourced from outside of the aircraft, typically a service truck. The inlet port 310 and the dispersant nozzles 320 are connected to each other through the appropriate plumbing 330. The system is controlled by controller 306 having appropriate switches, pumps, and the like. The disinfecting solution is pumped through nozzles 320 disperse the disinfecting solution throughout the aircraft fuselage.

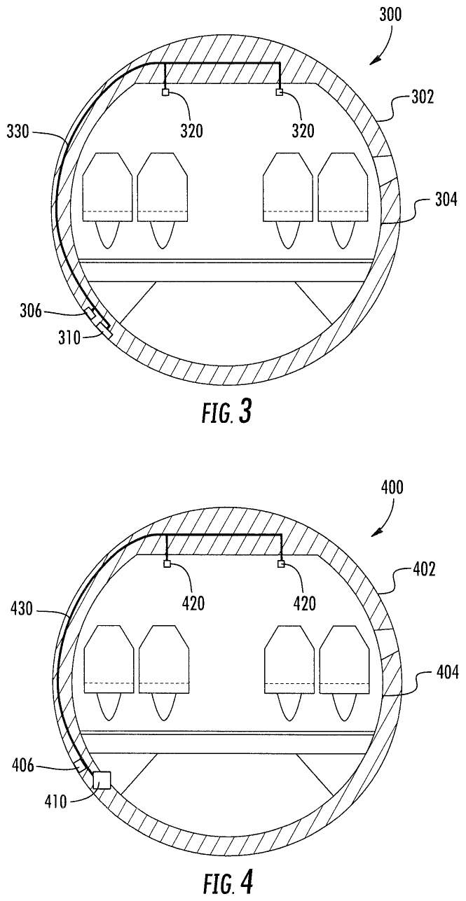

FIG. 4 is a cross-sectional view a further embodiment of the present invention showing an aircraft fuselage 400 having an outer shell 402 and an inner shell 404. There is provided a holding tank 410 located on the inside of aircraft fuselage 400 for holding disinfecting solution. The holding tank 410 is connected through plumbing 430 to dispersing nozzles 420. The system is controlled by controller 406 having appropriate switches, pumps, and the like.

Many modifications and other embodiments of the invention set forth herein will come to mind to one skilled in the which the invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for the purposes of limitation.

What is claimed is:

1. An aircraft disinfecting system for use in an aircraft fuselage having a frame, an inner shell and an outer shell comprising:
a source of disinfecting solution supplied to said aircraft;
plumbing inside of said aircraft fuselage between said inner shell and said outer shell connected to said disinfecting solution source for transferring said disinfecting solution;
disinfecting solution dispensers connected to the other end of said plumbing located inside of said aircraft fuselage for applying disinfecting solution to surfaces to be disinfected; and
a controller connected to said disinfecting system for controlling the supply of disinfecting solution throughout said aircraft fuselage.

2. The aircraft disinfecting system according to claim 1 wherein said source of disinfecting solution is an inlet port located on the outside of said the aircraft fuselage connecting said source of disinfecting solution to said inlet port and said disinfecting solution dispensers comprise dispersing nozzles connected to said plumbing and being located inside of said aircraft ventilation system.

3. The aircraft disinfecting system according to claim 1 wherein said source of disinfecting solution is an inlet port on the outside of said the aircraft fuselage connecting said source of disinfecting solution to said inlet port.

4. The aircraft disinfecting system according to claim 1 wherein said disinfecting solution dispensers comprise dispersing nozzles connected to said plumbing located inside of said aircraft ventilation system.

5. The aircraft disinfecting system according to claim 1 further comprising an electrostatic spray.

6. The aircraft disinfecting system according to claim 1 wherein said source of disinfecting solution is a holding tank located within said aircraft fuselage for holding a disinfecting solution and said disinfecting solution dispensers comprise dispersing nozzles connected to said plumbing located inside of said aircraft ventilation system.

7. The aircraft disinfecting system according to claim 1 wherein said source of disinfecting solution is a holding tank located within said aircraft fuselage for holding disinfecting solution.

8. The aircraft disinfecting system according to claim 1 wherein said source of disinfecting solution is a holding tank located within said aircraft fuselage for holding a disinfecting solution and said disinfecting solution dispensers are dispersing nozzles connected to said plumbing located throughout said aircraft.

9. The aircraft disinfecting system according to claim 1 wherein said a source of disinfecting solution supplied to said aircraft is an inlet port located on the outside of said the aircraft fuselage connecting a source of disinfecting solution to the aircraft and disinfecting solution dispensers connected to said plumbing comprise dispersing nozzles connected to said plumbing, said dispersing nozzles being located throughout the inside of said aircraft.

10. The aircraft disinfecting system according to claim 1 wherein said disinfecting solution dispensers are dispersing nozzles connected to said plumbing located throughout said aircraft.
